(19) United States
(12) Patent Application Publication
(10) Pub. No.: US 2010/0031730 A1
(43) Pub. Date: Feb. 11, 2010

(54) DEVICE FOR MEASURING THE AMMONIA CONTENT IN A GAS MIXTURE

(75) Inventors: Iris Van Uitert, Enschede (NL); Wouter Olthuis, Enschede (NL); Albert Van Den Berg, Nijverdal (NL)

Correspondence Address: RENNER OTTO BOISSELLE & SKLAR, LLP, 1621 EUCLID AVENUE, NINETEENTH FLOOR, CLEVELAND, OH, 44115, US

(73) Assignee: UNIVERSITEIT TWENTE a University

(21) Appl. No.: 12/513,890

(22) PCT Filed: Nov. 9, 2007
(86) PCT No.: PCT/NL2007/050551

§ 371 Date: Oct. 6, 2009

(30) Foreign Application Priority Data
Nov. 9, 2006 (NL) 2000310

(51) Int. Cl. G01N 33/497; G01F 1/00

(52) U.S. Cl. 73/23.3; 73/861; 73/1.06

(57) ABSTRACT

The invention relates to a device for measuring the ammonia content in a gas mixture. The device comprises at least one inlet and one outlet for the gas mixture, between which is situated an ammonia measuring system connected thereto. The device also comprises first regulating means for regulating the gas mixture flow through the inlet, and second means which are adapted such that they allow a measurement of the ammonia content which is substantially independent of the gas mixture flow rate. The device is preferably applied for measuring the ammonia content in exhaled air.

The invention relates to a device for measuring the ammonia content in a gas mixture, and in particular in exhaled air.

The measurement of the ammonia content in the breath of a person can be important in many medical applications. It is thus known for instance that measuring the ammonia content in the breath produces diagnostic information about patients suffering from a disturbed urea balance. Such a disturbed urea balance occurs as a result of kidney disorders or due to bacteriological stomach infections which can ultimately result in a gastric ulcer. The ammonia content in blood can also be of importance in the sports world. This is because during physical activity the body produces ammonia, wherein the production increases exponentially with the activity. If the ammonia content in the blood comes to lie at a higher level and herein rises above the average ammonia content in the environment, ammonia will diffuse from the blood to the lungs. Typical ammonia contents in exhaled air are here relatively low, and generally in the order of magnitude of 0.1 to 10 ppm (parts per million). Other gases are also present in the exhaled air in addition to ammonia. Exhaled air thus comprises for instance CO₂ in volumes up to about 3%. The presence of these gases makes an accurate measurement of the relatively low ammonia contents in the breath more difficult.

The object of the present invention is to provide a device for measuring the ammonia content in a gas mixture, in particular in exhaled air, which device is able to measure relatively low ammonia contents in accurate manner. There is further a need for a device for measuring the ammonia content in a gas mixture which does not take up much space and which can be readily arranged on a test subject.

The invention provides for this purpose a device of the type stated in claim 1, which device comprises at least one inlet and one outlet for the gas mixture, between which is situated a feed channel and an ammonia measuring system connected thereto, wherein the device also comprises first regulating means for regulating the gas mixture flow through the inlet, and second means which are adapted such that they allow a measurement of the ammonia content which is substantially independent of the gas mixture flow rate. It has been found that said measures enable an accurate measurement of relatively low ammonia contents in a gas mixture flow, even when the starting point is a gas mixture flow which has in principle a high flow rate and which is moreover not constant through time. This is for instance the case for a gas mixture flow in the form of exhaled air. Such a flow is not only interrupted during inhalation, but moreover has a typical flow rate of about 7000 ml/min., this corresponding to an average volume of 500 ml per exhalation. It is almost impossible for a person to exhale at a constant flow rate, so a considerable variation occurs around the average volume of 500 ml. By providing the device with first means it is possible to ensure that a predetermined volume of gas mixture is received per exhalation by the inlet in the feed channel.

The device can in principle comprise any suitable ammonia measuring system. Preferably however, the device is provided with a miniature ammonia measuring system, for instance in the form of a chip. Owing to the small dimensions thereof, a device comprising such a sensor can be easily arranged. It is thus possible for instance to attach a device according to this preferred variant to a test subject by accommodating this device in a support construction which can be arranged at the position of the mouth. It is also possible to accommodate the present preferred variant of the device in a carrying construction, for instance in a bag.

A particularly suitable ammonia measuring system comprises substantially three components: a sampling unit for the gas mixture, a selecting unit and a detecting unit. Via a microporous, water-repellent but gas-permeable membrane the gas mixture for analysis is brought into contact in the sampling unit with an acid in which a part of the gas mixture is dissolved. Due to the acid environment in the sampling unit and the high solubility of ammonia, very small quantities of ammonia will also be readily converted herein into ammonia ions. This is less the case for molecules present in the gas mixture which are less readily soluble, such as for instance CO₂ in exhaled air. The thus formed acidic gas mixture solution is pumped to the selecting unit which likewise comprises two spaces separated by a membrane. Water which has been purified (of other ions) is circulated in one of the spaces. Located in the other space is the acidic gas mixture solution to which a suitable basic solution is added. The ammonia ions present in the solution are thus neutralized to form ammonia gas which at least partially diffuses through the membrane to the water flow present in the other space The other molecules present in the solution will substantially ionize and not diffuse through the membrane.

The selecting unit therefore ensures that substantially ammonia enters the water flow. The detecting unit finally comprises an electrolytic conductivity sensor which detects the content of ammonia ions. A higher concentration of ammonia ions in the water results here in a higher conductivity.

A preferred embodiment of the device according to the invention is characterized in that the second means comprise a flow meter which measures the gas mixture flow rate to the ammonia measuring system, and correction means which correct the measured ammonia content for the measured flow rate. It has been found that the measurement of the ammonia content, and in particular of relatively low ammonia contents, greatly depends on the gas mixture flow rate. A suitable manner of displaying this dependence is by means of a calibration curve of ammonia content against gas mixture flow rate. Suitable correction means according to the invention comprise a computer which, using knowledge of the predetermined dependence of the measured ammonia content on the flow rate of the gas mixture for testing and with knowledge of the measured gas mixture flow rate, is able to determine the ammonia content for a determined gas mixture flow rate. What the desired gas mixture flow rate is depends on the specific conditions of the measurement. For an application in which the ammonia content of exhaled air is determined, a flow rate of about 50 ml/min is generally chosen, although this is not essential for the invention.

In another preferred embodiment of the device according to the invention the device comprises second means in the form of pump means for feeding the gas mixture at almost constant flow rate to the ammonia measuring system. The first means ensure that a determined volume of exhaled air is received in the feed channel per exhalation and inhalation cycle, after which the constant flow rate pump means ensure that a practically constant flow rate of gas mixture is fed to the ammonia measuring system. The present preferred variant has the advantage of no longer requiring correction means. Furthermore, the desired measuring flow rate can be adjusted in simple manner. Pump means are per se known, also for miniature devices. Suitable pump means comprise for instance an electromagnetic or membrane pump.

Yet another preferred embodiment of the inventive device is characterized in that the second means comprise pressure-regulating means which regulate the pressure difference over the ammonia measuring system. Such a preferred variant has the advantage that it does not necessarily have to comprise constant flow rate pump means in order to enable feeding of a practically constant gas mixture flow rate to the ammonia measuring system. This makes the device simpler, and therefore also more reliable. The feed channel between the inlet and the ammonia measuring system is preferably provided with at least one branch with outlet. The feed channel is more preferably provided downstream of the branch with a (second) control valve. There are advantages to the first regulating means also comprising a (first) control valve. According to the invention the pressure-regulated second valve accommodated in the feed channel for the gas mixture flow will open to the outlet if an increased pressure level resulting from exhaled air is reached in said channel. The pressure-regulated second valve is preferably coupled to the first control valve in the channel between the inlet and the ammonia measuring system. The first control valve opens and carries gas mixture, preferably via a flow constriction, out of the feed channel over the ammonia measuring system as soon as and as long as the pressure-regulated second valve is also open. This provides an almost constant pressure in the feed channel, and thereby also an almost constant flow rate of the gas mixture flow in the ammonia measuring system.

In yet another preferred embodiment the device is provided with a flow meter upstream of the first regulating means. The device is preferably also provided with a buffer space in which the gas mixture can be temporarily stored, wherein the buffer space is preferably situated between the inlet and the at least one branch. Such a buffer space ensures that a determined volume of exhaled air can be temporarily stored. Preferably providing the buffer space upstream and downstream with control valves provides the option of temporarily storing the volume of air exhaled in one exhalation in the buffer space and guiding it therefrom at almost constant flow rate to the ammonia measuring system. A mixing of multiple exhalation volumes is hereby avoided, which further enhances the accuracy of the measurement.

There are further advantages in characterizing the device according to the invention in that the gas mixture comprises exhaled air, and that the device is also provided with supply means for the exhaled air connectable to the inlet. It hereby becomes possible to feed exhaled air in substantially controlled manner to the device, and in particular to the ammonia measuring system. The supply means preferably comprise a flexible tube, if desired provided with a suitable mouthpiece.

Yet another preferred embodiment of the device also comprises heating means for at least the ammonia measuring system. It is thus possible for instance to arrange the device in a heatable holder. Providing the device with heating means reduces or even avoids condensation of the exhaled air. This further enhances an accurate measurement of the ammonia content. It is also advantageous if the supply means are also heated. The device is preferably provided for this purpose with heating means in the form of a resistance wire connectable to a power source. Heating to a temperature at which condensation is substantially avoided is in principle already sufficient, wherein the precise temperature will depend, among other factors, on the temperature and the degree of humility of the environment. It is however advantageous for the heating means to also comprise a temperature regulator. Using such a regulator the desired temperature of the device, or at least parts thereof, can be set to the predetermined, most suitable level. It has been found that in the case of a device for measuring the ammonia content in exhaled air, wherein use is made of supply means in the form of a flexible tube, the most suitable temperature is a few degrees higher than the body temperature, preferably up to 10° C. higher, still more preferably up to 5° C. higher.

The device according to the invention can be used for many purposes. It is thus possible to use the device to measure the ammonia content in the environment, for instance in the vicinity of factories, or in highly urbanized areas. The device is however preferably applied for measuring the ammonia content in the airflow exhaled by a person.

The advantages of the device according to the invention become particularly manifest when the person is involved in physical exertion and the ammonia content is measured during this exertion. It has been found that the device is particularly suitable for determining the anaerobic limit by means of a measurement of the ammonia content. The human body consists for the greater part of carbon, hydrogen and nitrogen. These elements are generally present in the form of amino acids and sugars. In order to function properly the body requires energy, and this is obtained by combustion processes in which particularly oxygen is consumed and carbon dioxide is produced. Amino acids are also degraded during the combustion, wherein ammonia is formed. In normal conditions the ammonia content in the body is very low, in the order of magnitude of 30 μmol/l. During physical exertion however this content can rise to 100-200 μmol/l if the anaerobic limit is exceeded during this exertion. This relatively sudden rise in the ammonia content is attributed to lactate production in the muscles. When the anaerobic limit is exceeded a sportsperson rapidly becomes tired and he/she will have to slow down. Furthermore, training below the anaerobic limit is more effective than doing so above it. Determining the personal anaerobic limit is therefore of essential importance for a sportsperson. The anaerobic limit is determined according to the prior art by measuring the lactate content in the blood. This known method requires successive taking of blood, for instance every three minutes, and this is a highly taxing for the athlete. An accurate and less taxing method is provided according to the invention by determining the anaerobic limit by measuring the ammonia content in the breath.

The invention will now be elucidated on the basis of non-limitative exemplary embodiments shown in the following figures. Herein:

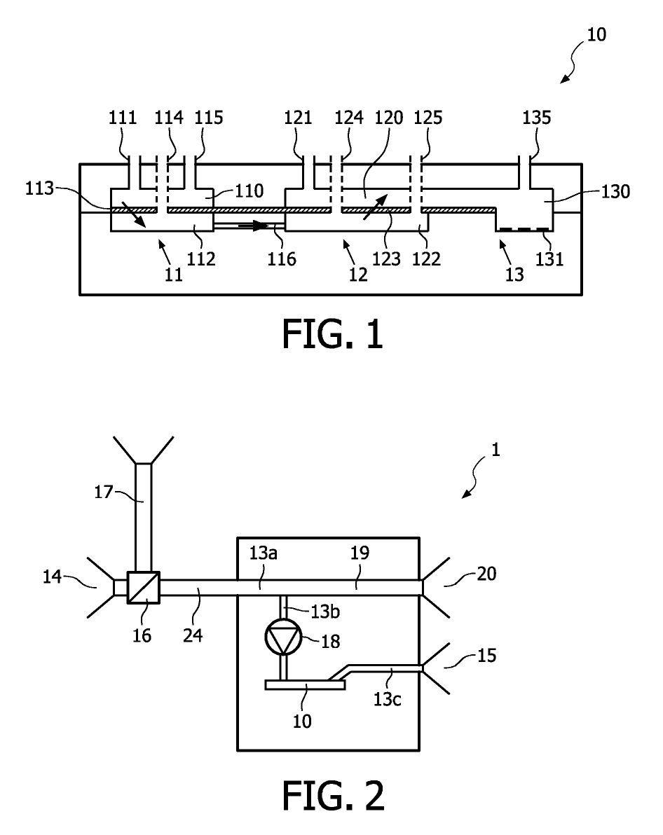

FIG. 1 shows schematically an ammonia measuring system as applied in the device according to the invention;

FIG. 2 shows schematically a first exemplary embodiment of the device according to the invention;

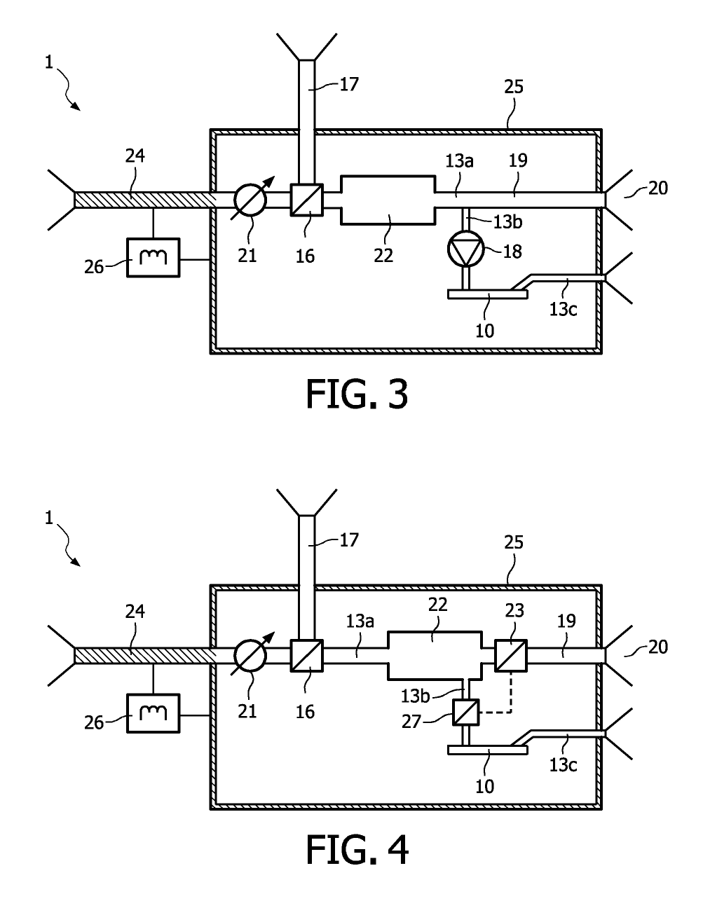

FIG. 3 shows schematically a second exemplary embodiment of the device according to the invention;

FIG. 4 shows schematically a third exemplary embodiment of the device according to the invention; and

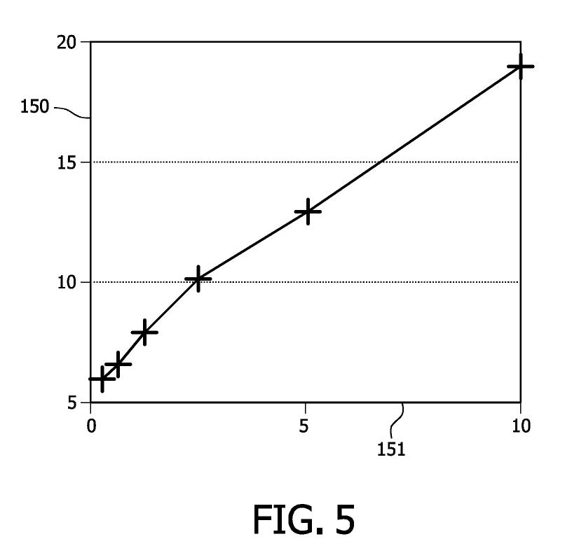

FIG. 5 finally shows a graphic representation of the conductivity as a function of the ammonia content present in the gas mixture.

Referring to FIG. 1, a miniature ammonia measuring system 10 is shown which is applied in the device according to the invention. Although the invention is not limited hereto, the operation of the device and ammonia measuring system 10 will be elucidated with reference to the exemplary embodiments described hereinbelow for measuring the ammonia content in exhaled air. Such a gas mixture generally comprises at least oxygen, CO₂ and a low ammonia content. Ammonia measuring system 10 comprises substantially three components: a sampling unit 11 for the gas mixture, a selecting unit 12 and a detecting unit 13. Sampling unit 11 comprises a space 110 into which the gas mixture for analysis—in this case exhaled air—is pumped via a supply conduit 111. A second space 112 contains a solution of the gas mixture in a suitable acid, for instance NaHSO₄. The acid is pumped into space 112 via supply conduit 114. The two spaces (110, 112) are separated by means of a microporous, water-repellent but gas-permeable membrane 113. Because the gas mixture can diffuse through membrane 113, an equilibrium is formed between the dissolved state in space 112 and the gas phase in space 110. Excess gas mixture can optionally be discharged via discharge conduit 115. Owing to the acidic environment in space 112 and the high solubility of ammonia, very small quantities of ammonia will also be readily converted herein into ammonia ions. This is less the case for the CO₂ which is likewise present in the gas mixture and which is considerably less readily soluble. The thus formed acidic gas mixture solution is pumped to selecting unit 12 via conduit 116. Selecting unit 12 likewise comprises two spaces (120, 122). In space 120 water that has been purified (of other ions) is circulated via supply conduit 121. Space 122 contains the acidic gas mixture solution to which a suitable basic solution, for instance a 0.25 M NaOH solution, is added via supply conduit 124. The pH of the solution is thus increased, for instance to a pH=13. The ammonia ions present in the solution are thus neutralized to form ammonia gas which at least partially diffuses through a second membrane 123 to the water flow present in space 120. The CO₂ present in the solution will substantially ionize however, and is discharged via discharge conduit 125. Selecting unit 12 thus ensures that it is substantially ammonia which enters the water flow. Detecting unit 13 comprises a space 130 in which purified water with ammonia gas is situated. The ammonia gas will at least partially react with the water and form ions. Excess water can be drained via conduit 135. Space 130 also comprises an electrolytic conductivity sensor 131. The conductivity measured with sensor 131 depends on, among other factors, the conductivity of the electrolyte and on the cell constant of detecting unit 13. The conductivity of the electrolyte is the product of the content (expressed in mol/m³) of all ions present in the electrolyte and the conductivity of these ions. A higher concentration of ammonia ions in the water will thus result in a higher conductivity. An example of the relation between the ammonia content in a gas mixture and the conductivity measured with sensor 131 is shown in FIG. 5. The conductivity 150 (measured in μS) shown along the y-axis has a non-linear increasing relation with the ammonia content 151 in the gas mixture (measured in μM) shown along the x-axis. Such a curve can be used to convert a measurement of the conductivity 150 into a measurement of the ammonia content 151 in the gas mixture. The shown sensor 10 is able to measure relatively low ammonia contents, preferably below ppb level (ppb=parts per billion).

Referring to FIG. 2, a first exemplary embodiment of device 1 according to the invention is shown. Device 1 comprises at least one inlet 14 and at least one outlet 15 for the gas mixture. Situated between inlet 14 and outlet 15 is an ammonia measuring system 10 of the type as described above. Ammonia measuring system 10 is connected to inlet 14 and outlet 15 via a system 16 of feed conduits, through which the gas mixture for analysis can be carried. It will be apparent that device 1 is provided with all auxiliary means, such as for instance feed and discharge conduits, necessary for good operation of ammonia measuring system 10. These auxiliary means are not shown in detail in FIGS. 2-4. For a description hereof reference is made to the description relating to FIG. 1. In the shown exemplary embodiment device 1 is equipped for measuring the ammonia content in exhaled air. Inlet 14 is provided for this purpose with an appropriate mouthpiece. Device 1 is further provided with the first regulating means in the form of a control valve 16. Control valve 16 regulates the gas mixture flow which is blown from inlet 14 into conduit system 13. Control valve 16 is also connected to a discharge conduit 17 along which excess exhaled air can escape in an open position of control valve 16 to conduit 17. Ambient air can also be drawn in via conduit 17. Device 1 is further provided according to the invention with a pump 18, for instance an electromagnetic pump or a membrane pump. In an open position of control valve 16 to conduit system 13 the exhaled air is carried by means of pump 18 via this conduit system 13 to the ammonia measuring system where it enters via inlet 111 (see FIG. 1). Pump 18 is configured such that it can supply a practically constant flow rate of the gas mixture to ammonia measuring system 10. Owing to this measure the measurement of the ammonia content becomes markedly more accurate, whereby measurement becomes possible of ammonia contents lower than 1 ppm, preferably lower than 100 ppb, still more preferably lower than 1 ppb. Device 1 further comprises at least one branch 19 with outlet 20 to the environment. If desired, excess gas mixture can be discharged via this branch 19. Device 1 operates substantially as follows. In the opened position of control valve 16 a flow of air will be maintained through conduit 13a and branch 19 when a test subject blows in air via inlet 14. Although greatly dependent on the test subject, a typical flow rate during exhalation is in the order of magnitude of 7000-8000 ml/min. This is not however a constant flow since inhalation and exhalation take place an average of 13-15 times per minute. An average flow rate of about 500 ml is thus reached per exhalation. A part of this airflow will leave the device again via discharge 20. Pump 18 ensures that a part of the airflow is taken off and fed at practically constant flow rate via conduit 13b to the ammonia measuring system 10. Although the invention is in no way limited hereby, it has been found that good results are achieved with an average flow rate lying between 10 and 100 ml/min, preferably between 25 and 80 ml/min and most preferably between 35 and 65 ml/min. After being analyzed, the gas mixture leaves the device via conduit 13c and discharge 15.

A second embodiment of device 1 according to the invention is shown in FIG. 3. The shown embodiment variant differs from the variant shown in FIG. 2 in that it is provided upstream of control valve 16 with a flow meter 21. A buffer space 22 is further arranged in conduit system 13. In the shown variant this is situated upstream of branch 19, although this is not essential. The device according to the preferred variant shown in FIG. 4 can also be further provided with a second control valve 23 situated downstream of buffer space 22 in branch 19. The above-mentioned components are held together by enclosing thereof in a holder 25. Exhaled air is fed to holder 25 via supply means 24 for the exhaled air connectable to the inlet. Supply means 24 can for instance comprise a flexible tube or hose of determined length provided at one outer end with a mouthpiece. The test subject can in this way blow in air relatively easily without being impeded by other components of the device which, if desired, can be placed at a distance. In order to prevent condensation of at least ammonia measuring system 10, device 1 is preferably also provided with heating means 26, for instance in the form of a resistance wire connectable to a power source. In the preferred variants shown in FIGS. 3 and 4 at least the hatched portion—flexible supply conduit 24 and holder 25—is heated. It is however also possible to heat fewer components, such as for instance only ammonia measuring system 10. It is advantageous to heat the heated components substantially to body temperature or, if desired, to slightly higher temperature, preferably up to 10° C. higher than body temperature, still more preferably up to 5° C. higher. In order to further improve the ammonia measurement, the device preferably also comprises heating means 26 which also comprise a temperature regulator (not separately shown). The embodiment shown in FIG. 3 operates substantially as follows. In the opened position of control valve 16 a flow of air will enter buffer space 22 when a test subject blows in air via supply conduit 24. A part of this airflow will leave the device again through conduit portions 13a and 19 and via discharge 20. Pump 18 ensures that a part of the airflow is taken off and fed at practically constant flow rate via conduit 13b to ammonia measuring system 10. After being analyzed, the gas mixture leaves the device via conduit 13c and discharge 15. During exhalation the flow rate of the exhaled airflow is measured using flow meter 21. Using this measurement the opening time necessary to fill buffer space 22 once with replenished air is computed for control valve 16. At the moment buffer space 22 is filled with replenished air control valve 16 is closed and possible excess breathing air is discharged via conduit 17 to the environment. The replenished air present in buffer space 22 is pumped to ammonia measuring system 10 using the constant flow rate pump 18. In a further preferred variant the conduit 19 is provided downstream of buffer 22 with a second control valve 23 (not shown in FIG. 3). At the moment that the test subject breathes out again, this is detected by flow meter 21. This then actuates control valve 23 so that it opens. The air still present in buffer space 22 can hereby be expelled via discharge 20. The air in the buffer space is thus replaced by replenished air, after which both control valves (16, 23) close. This variant has the additional advantage that air can also be pumped during inhalation to ammonia measuring system 10 using pump 18, so that a continuous measurement is possible. Furthermore, measurement always takes place on replenished air, which further enhances the accuracy of the measurement. This is not possible in the variant shown in FIG. 1. This is because this variant has the drawback that a constant airflow rate is not continuously available. The sampling time must then preferably be less than or equal to the period in which the practically constant flow rate is available.

Yet another preferred variant is shown in FIG. 4. Use is not made in this variant of a constant flow rate pump. In the shown device the buffer space 22 is provided with a pressure-regulated valve 23 which opens (to discharge 20) when a determined pressure (as a result of exhaled air) is reached in buffer space 22. This pressure-regulated valve 23 is coupled to a control valve 27 which is accommodated in conduit 13b to ammonia measuring system 10 and which is opened as soon as and as long as the pressure-regulated valve 23 is also open. In this way an almost constant pressure is obtained in buffer space 22. In the open position of valve 27 the air is preferably carried via a constriction (not shown) out of buffer space 22 and over ammonia measuring system 10. An almost constant flow rate in the ammonia measuring system is likewise achieved in this manner without a constant flow rate pump 18 being necessary for this purpose.

Yet another preferred variant comprises a device 1 in which flow meter 21 is used to measure the gas mixture flow rate to ammonia measuring system 10. The device further comprises correction means (not shown) which correct the measured ammonia content for the measured flow rate. This takes place on the basis of the predetermined dependence of the measured ammonia content on the flow rate of the gas mixture to be tested. An appropriate method of displaying this dependence is by means of a calibration curve of ammonia content against gas mixture flow rate.

It will be apparent that the invention is not limited to the exemplary embodiments shown and described here, but that within the scope of the appended claims numerous variants are possible which will be self-evident to the skilled person in this field.

1. Device for measuring the ammonia content in a gas mixture, which device comprises at least one inlet and one outlet for the gas mixture, between which is situated a feed channel and an ammonia measuring system connected thereto, wherein the device also comprises first regulating means for regulating the gas mixture flow through the inlet, and second means which are adapted such that they allow a measurement of the ammonia content which is substantially independent of the gas mixture flow rate.

2. Device as claimed in claim 1, wherein the second means comprise a flow meter which measures the gas mixture flow rate to the ammonia measuring system, and correction means which correct the measured ammonia content for the measured flow rate.

3. Device as claimed in claim 1, wherein the second means comprise pump means for feeding the gas mixture at almost constant flow rate to the ammonia measuring system.

4. Device as claimed in claim 1, wherein the second means comprise pressure-regulating means which regulate the pressure difference over the ammonia measuring system.

5. Device as claimed in claim 1, wherein it is provided between the inlet and the ammonia measuring system with at least one branch with outlet.

6. Device as claimed in claim 1, wherein it is provided with a flow meter upstream of the first regulating means.

7. Device as claimed in claim 1, wherein the first regulating means comprise a control valve.

8. Device as claimed in claim 5, wherein it is provided downstream of the branch with a control valve.

9. Device as claimed in claim 1, wherein it is also provided with a buffer space in which the gas mixture can be temporarily stored.

10. Device as claimed in claim 9, wherein the buffer space is situated between the inlet and the at least one branch.

11. Device as claimed in claim 9, wherein the device also comprises second regulating means between the buffer space and the at least one buffer space outlet.

12. Device as claimed in claim 1, wherein the gas mixture comprises exhaled air, and that the device is also provided with supply means for the exhaled air connectable to the inlet.

13. Device as claimed in claim 12, wherein the supply means comprise a flexible tube.

14. Device as claimed in claim 1, wherein the device also comprises heating means for at least the ammonia measuring system.

15. Device as claimed in claim 14, wherein the heating means comprise a resistance wire connectable to a power source.

16. Device as claimed in claim 14, wherein the heating means also comprise a temperature regulator.

17. A method for measuring the ammonia content in the airflow exhaled by a person comprising delivering the air exhaled by the person to the inlet of the device as claimed in claim 1.

18. The method as claimed in claim 17, wherein the person is subjected to physical exertion and the ammonia content is measured during this exertion.

19. The method as claimed in claim 18, wherein the anaerobic limit is determined from the measured ammonia content.
